(19) United States
(12) Patent Application Publication
(10) Pub. No.: US 2003/0028137 A1
(43) Pub. Date: Feb. 6, 2003

(54) Novel hypothermic modalities and direct application of protective agents to neural structures or into CSF

(76) Inventor: Bruce H. Levin, Merion Station, PA (US)

Correspondence Address: Bruce H. Levin MD, 125 Broome Lane, Merion Station, PA, 19066, US

(21) Appl. No.: 10/150,538

(22) Filed: May 17, 2002

Related U.S. Application Data
(60) Provisional application No. 60/292,027, filed on May 18, 2001.

(51) Int. Cl. A61M 5/00

(52) U.S. Cl. 604/8; 607/96

(57) ABSTRACT

Novel use of CNS hypothermia and csf drug delivery to minimize CNS insult in a variety of disease states is disclosed. Specifically, cooling of CSF, epidural or subdural spaces are discussed and the direct delivery of pharmacologically active agents directly into the CSF to improve delivery to damaged or vulnerable CNS tissues if disclosed.

### CROSS REFERENCE TO RELATED APPLICATION

This application claims the filing benefit under 35 U.S.C. §119(e) of U.S. Provisional Application No. 60/292,027, filed May 18, 2001, which is included herein by reference.

### TECHNICAL FIELD

The present invention pertains generally to neuroprotection, and more particularly to novel methods of cooling CNS structures in a more effective manner or delivering neuroprotective agents into areas of Cerebrospinal fluid circulation to affect improved neuroprotection.

### BACKGROUND OF THE INVENTION

Hypothermia has been proven to decrease the metabolic demands of tissues and organs and in a variety of settings. CNS structures are particularly vulnerable to hypoperfusion, hypoxia and other insults. Intellectually, hypothermia is an extremely attractive methodology for increasing brain or spinal cord tissue survival in many clinical scenarios. However, many obstacles have prevented its use in this regard. Notably, generalized hypothermia has significant affects on many organ systems and these may create significant problems. For example, cardiac conduction and contractility are adversely affected below certain thresholds, as are blood viscosity and coaguabilty. Respiration may cease and mechanical ventilation maybe required. Furthermore, techniques used to cool the brain or spinal cord have not been particularly effective. For example, applying external cooling devices to the head is not optimally effective because the vascularity of the scalp and the insulation provided by the structures therein limit direct cooling. Cooling the arterial inflow can be effective, but the rapid flow of blood through a relatively small cross sectional area located within a larger section of tissues presents many limitations. CNS hypothermia has been attempted by cooling blood entering the CNS but this introduces the many problems associated with the resulting cardiac and systemic hypothermia. Thus there is no effective way to cool the CNS to a much greater relative degree than than the rest of the body. This limits the effectiveness of current attempts to provide clinically useful CNS hypothermia.

### SUMMARY OF THE INVENTION

The current invention discloses the use of hypothermia to decrease central nervous system global ischemic, hypoxic, toxic or metabolic tissue damage by the use of hypothermia accomplished by insertion of cryoprobe, or introduction of cooled fluids into the cerebral spinal fluid or epidural or subdural space or by shunt or recirculation of externally cooled cerebrospinal fluid into the subarachnoid or ventricular areas. The use of the epidural or subdural or subarachnoid or ventricular routes for the introduction of pharmacologically active agents or substrates to decrease ischemic, hypoxic, toxic or metabolicly induced damage to central nervous system structures and tissues is also disclosed. Central nervous system Arterial inflow cooling with the possibility of post cerebral circulation rewarming and arterial instillation of neuroprotective agents is also disclosed.

Other features and advantages of the present invention will become apparent from the following detailed description, taken in conjunction with the accompanying drawings, which illustrate, by way of example, the principles of the invention.

### BRIEF DESCRIPTION OF DIAGRAMS

Figure A. Illustrates normal flow of CSF. Access into the CSF can be made at any point along its course.

Figure B. Illustrates one method of accessing the CSF, in this case intraventricularly, although the catheter or probe may be placed in any area where CSF circulates or in the epidural, subdural, or other place in close proximity to CSF flow. Figure B (1) represents a single or multi lumen, single or multiport catheter through which CSF may be cooled and recirculated or through which cooled fluid or CSF may be introduced and which medications or other neuroprotective agents may be introduced. Alternatively, figure B (1) may represent a cooling device such as a cryoprobe with or without an infusion port for medication delivery. Figure B (2) shows the catheter sealed in place. Figure B (3) represents a valve which may be used to regulate flow. Figure B (4) shows a catheter going external to the body to enter cooling unit or fluid or drug reservoir.

### DETAILED DISCRIPTION OF THE INVENTION

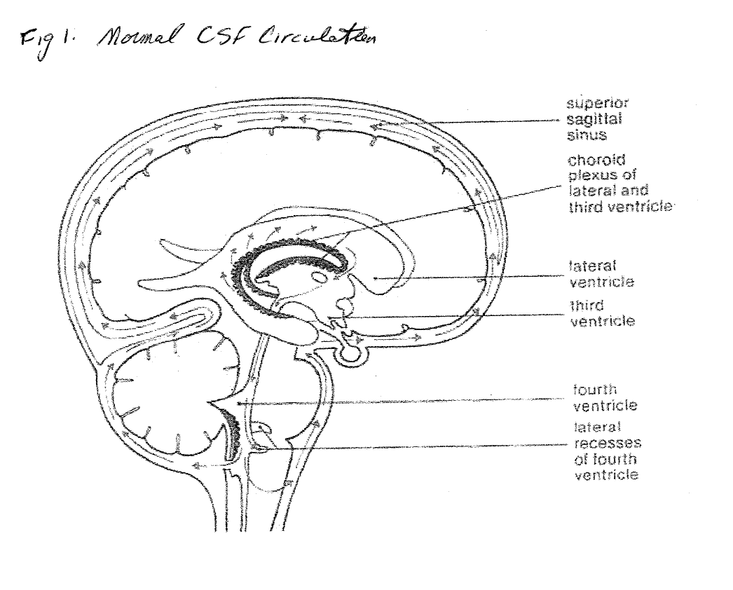

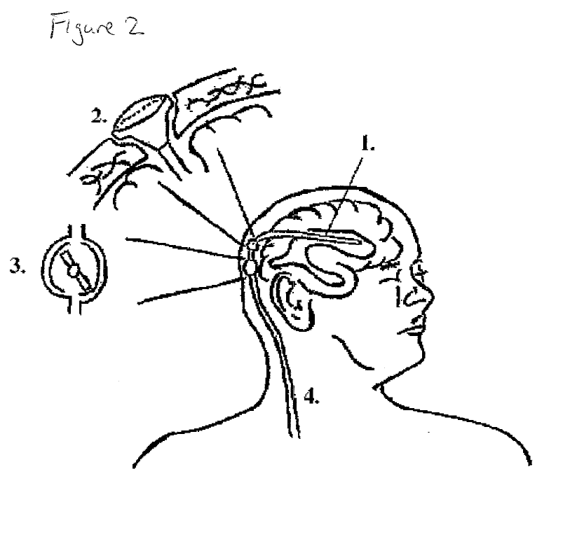

The present invention is directed to novel methods of cooling CNS structures in a more effective manner. Of course, generalized hypothermia or other modalities may be utilized in conjunction with the methods of this invention. Please refer to FIG. 1 which illustrates CNS anatomy and CSF flow in the human brain, spinal cord and related structures. Invasively accessing the epidural, subdural, intrathecal, or other related spaces or structures offers many potential advantages. Often, ventricular-peritoneal shunts are placed to drain CSF, or intracranial monitors are placed to measure the pressure within the cranial vault. These procedures are common and relatively safe, and present interesting possibilities for inducing CNS hypothermia. For example, a cryoprobe, or other cooling device, can be introduced, with techniques similar to portions of either of these procedures, to cool intracranial structures. Alternatively, the cooling device may be included or inserted in the manner of a spinal level epidural or intrathecal catheter or similar device. Furthermore, a device which would circulate cold fluid directly epidurally or into areas where CSF normally flows could be utilized, or CSF could be extracted or otherwise cooled, and then recirculated. A pressure monitoring system could be included to avoid adverse sequelae, and volumes of infused and extracted fluid as well as other relevant volumes or pressures or flow rates could be measured and regulated. Note that iatrogenic complications have been described from high pressure flushes improperly hooked to intracranial monitoring devices, causing intracranial hypertension and brain herniation. In one embodiment, CSF could be extracted, cooled and recirculated with or without drugs, oxygenated media, antioxidents, membrane stabilizers, energy substrates and the like. It would also be possible that partial or total reversal of CSF flow would allow better cooling of deep brain structures. Because of the high specific heat of water, aqueous fluids are very effective cooling media. Therefore, bathed tissues could be cooled considerably. Note that the CSF circulates in many areas that may be particularly vulnerable to watershed ischemia, and that ischemic tissues by definition are difficult to access by a vascular route because they get little if any blood flow. Therefore using a vascular based approach for cooling or medication delivery is suboptimal, and using direct cooling of the intrathecal/epidural or other neural compartments via the techniques of this invention would be more effective.

These same techniques can be used to provide spinal cord hypothermia, and cooling CSF located at the level of the sacral, lumbar, thoracic or cervical spine may provide some degree of limited brain hypothermia as well, depending upon flow characteristics. Also interesting to note is that the venous drainage of CSF could provide cooling of areas of the brain in proximity to the sagittal sinus and related veins.

Many of the characteristics of CSF flow which make invasive hypothermia alluring also hold out promise for other therapies as well. For example, barbiturates, membrane stabilizers, local anesthetics, magnesium and other pharmacologic agents may be limited in their clinical effectiveness in neuroprotection by their adverse cardiovascular profile. Also, blood borne agents maybe unlikely to be delivered, to their ischemic target areas. Perhaps steroids, membrane stabilizers, ions such as magnesium, anticytokines, other antiinflammatory agents, antiseizure or other drugs delivered locally or to the CSF may have a greater therapeutic affect with less systemic affects. Therefore, drug delivery with this model could be rewarding clinically in a variety of settings. This method may also be combined with all currently utilized delivery methodologies of the same or different pharmacologic agents to provide optimal delivery to tissues at risk. Furthermore, providing energy substrates or even oxygenating a suitable infusate or the CSF could lead to improved tissue salvage, particularly in the vulnerable periventricular areas. In a preferred embodiment, magnesium, membrane stabilizing drugs such as dilantin, calcium channel blockers, local anesthetics and the like, as well as other agents to decrease metabolic demands would be utilized. In spinal cord injury intrathecal steroids, antiinflammatory and antiautoimmune agents, as well as drugs to inhibit scarring, could be used. Perhaps low dose agents such as rapimmune could be effective. It would be of benefit to combine hypothermia with drug introduction into the CSF or otherwise near neural structures.

In those instances where a vascular approach to CNS cooling is desired, a catheter may be placed into or near the desired vessel, for example, the carotid. A cryoprobe or cold fluid with or without pharmacologic agents could be introduced. If systemic hypothermia is problematic, typical heating methods may be used, or a catheter or heating probe placed in the central venous circulation could warm blood before it enters the heart, preventing dangerous temperature induced alterations of cardiac rhythm, contractility or overall function. A limited arterial-venous temperature differential would thus limit cardiac as well as systemic hypothermia and excess fluid could be removed by a simple dialysis circuit if needed. Alternatively, microwave, ultrasound or other heating modalities may be used to heat blood in the great vessels, lung or heart. Ideally, these modalities may be used in conjunction with monitoring modalities including but not limited to EEG, spectral analysis, intracranial blood flow, and other available monitors of tissue perfusion and ischemia.

Potential Applications

cardiac arrest

stroke, tia, rind

impending or worsening neurologic ischemia

intracranial hypertension

head or spine trauma

refractory seizure activities including status epilepticus and febrile seizures

hyperthermic states

intracerebral air embolus, decompression sickness (This should decrease both ischemic damage by decreasing metabolism while intravascular bubble size will decrease secondary to increased gas solubility at lower temperatures leading to decreased vessel occlusion.)

during high risk invasive procedures and surgeries

during poisonings with metabolic decouplers such as cyanide, or states adversely affecting oxygen utilization such as septic shock. (Since tissue oxygen utilization is limited by the toxins, decreasing consumption is attractive.)

other states placing the CNS at risk including impending or worsening hypoxia, hypertension or severe anemia

The preferred embodiments of the invention described herein are exemplary and numerous modifications and rearrangements can be readily envisioned to achieve an equivalent result, all of which are intended to be embraced within the scope of the appended claims

I claim:

1. A method of directly accessing Cerebrospinal fluid or tissues or spaces in close proximity to Cerebrospinal fluid with a catheter, probe, shunt or other appropriate device whereby hypothermia is induced in the Cerebral spinal fluid or related tissues or spaces to provide for cooling of central nervous system tissues or structures at risk of damage during periods of global neurologic hypoxia, hypoperfusion, or metabolic compromise, thereby reducing damage to these tissues or structures.

2. A method of claim 1 where neurologic global hypoxia, hypoperfusion, or metabolic compromise is the result of trauma, cardiovascular disease, cardiac, ischemia, dysrhythmia, failure or arrest, sepsis, pulmonary failure, hypoxemia, hemorrhage, anemia, poisoning, metabolic or endocrine compromise or other shock or shocklike states.

3. A method of claim 1 of limiting neurologic damage during seizures by inducing hypothermia

4. A method of claim 1 treating seizures by inducing global or regional hypothermia

5. A method of claim 1 limiting neurologic or other damage during sepsis by inducing hypothermia

6. A method of claim 1 decreasing neurologic damage during hyperthermic states by direct csf cooling

7. A method of claim 1 treating decompression sickness or air embolic phenomenae by inducing hypothermia

8. A method of claim 1 decreasing neurologic damage secondary to decompression sickness or gas or air embolic phenomenae by direct csf hypothermia

9. A Method of claim 1 treating intracranial hypertension or brain swelling or edema by inducing global or regional hypothermia.

10. A method of directly accessing Cerebrospinal fluid or tissues or spaces in close proximity to Cerebrospinal fluid with a catheter, shunt, probe or other drug infusion device to provide for delivery of therapeutic materials or pharmacologic agents directly to the cerebral spinal fluid or epidural, subdural or related tissues or spaces to affect a decrease in CNS tissue injury during periods of ischemic, hypoxic, metabolic, or toxic insult to the cental nervous system.

11. Method of claim 10 where the CSF, epidural or subdural space is accessed at the level of the spinal column.

12. Method of claim 10 where the CSF, epidural or subdural space is accessed through the cranium.

13. Method of claim 10 where the therapeutic or pharmacologic agent includes at least one of the following chosen from the group consisting of oxygen or oxygenated substrates, glucose metabolites, ATP, NADPH, lipids, fatty acids, Antioxidents, vitamins e, c, or b class, selenium, magnesium, local anesthetics, membrane stabilizers, calcium channel blockers, NMDA antagonists, antiseizure medications, anti-inflammatory agents, corticosteroids, barbiturates, benzodiazepines, anesthetic agents, agents to decrease metabolic rate, anticytokine agents, antibodies to inflammatory materials, anti tumor necrosis factor agents, antiscarring agents, rapimmune, or materials meant to bind toxins or toxic products of metabolism.

14. Method of claim 10 where hypothermia is induced

15. A Method of inducing CNS Hypothermia by cannulation of carotid or vertebral arterial inflow to provide a route of introduction for cryoprobe, cooled fluids or other hypothermic modalities.

16. A Method of accessing the carotid or vertebral artery circulation to allow introduction of pharmacologic or other agents to CNS structures without first pass or intial systemic dilution.

17. A Method of claim 16 which provides for warming of cooled blood returning from cooled CNS by introducing thermoprobe or warmed fluids to the central venous or atrial circulation.

18. A Method of claim 16 utilizing an arterial-venous shunt to allow cooled blood or fluids to enter the CNS arterial inflow while heating returning venous blood or fluids and reintroducing to the venous inflow.
